SEQUENCE LISTING (4 sequences)

SEQ ID NO: 1 - protein, 9 aa, synthetic construct
HLVEALYLV

SEQ ID NO: 2 - protein, 9 aa, synthetic construct
KIFGSLAFL

SEQ ID NO: 3 - protein, 9 aa, synthetic construct
NLVPMVATV

SEQ ID NO: 4 - protein, 9 aa, synthetic construct
GLCTLVAML

(19) United States
(12) Patent Application Publication
(10) Pub. No.: US 2025/0110122 A1
(43) Pub. Date: Apr. 3, 2025

(54) METHODS OF IDENTIFYING SUBJECTS RESPONSIVE TO TREATMENT FOR AN AUTOIMMUNE DISEASE AND COMPOSITIONS FOR TREATING THE SAME

(71) Applicant: The General Hospital Corporation, Boston, MA (US)

(72) Inventor: Denise L. FAUSTMAN, Boston, MA (US)

(21) Appl. No.: 18/978,388

(22) Filed: Dec. 12, 2024

Related U.S. Application Data
(63) Continuation of application No. 15/027,757, filed on Apr. 7, 2016, now Pat. No. 12,174,188.
(60) Provisional application No. 61/892,140, filed on Oct. 17, 2013.

(51) Int. Cl. G01N 33/566; A61K 39/00; A61K 39/04; A61K 45/06; G01N 33/50; G01N 33/564

(52) U.S. Cl. CPC G01N 33/566; A61K 39/04; A61K 45/06; G01N 33/505; G01N 33/564; A61K 2039/572; G01N 2333/47; G01N 2333/62; G01N 2333/70517; G01N 2800/042; G01N 2800/24; G01N 2800/52

(57) ABSTRACT

The invention features methods of identifying a subject having an autoimmune disease, such as type 1 diabetes, as likely to respond to treatment with a tumor necrosis factor-α (TNF-α) receptor II activator. The method involves measuring CD8 protein density on the surface of autoreactive CD⁸⁺ T cells and identifying the subject as likely to respond to the treatment if the CD8 protein density is reduced relative to a reference CD8⁺ T cell. For type 1 diabetes, the method may involve measuring C-peptide levels in an in vitro biological sample from the subject, identifying the subject as likely to respond to the treatment if the C-peptide levels are detectable, and identifying the subject as unlikely to respond to the treatment if the C-peptide are substantially undetectable. The invention also features pharmaceutical compositions of one or more TNFR2 activators for use in treating an autoimmune disease in a subject identified as likely to respond to the treatment prior to the treating by according to the methods of the invention.

### SEQUENCE LISTING

The instant application contains a Sequence Listing which has been submitted electronically in XML format and is hereby incorporated by reference in its entirety. Said XML copy, created on Dec. 11, 2024, is named “00786-569003_Sequence_Listing_12_11_24” and is 4,430 bytes in size.

### FIELD OF THE INVENTION

In general, the present invention relates to methods of identifying subjects having an autoimmune disease that are likely to respond to treatment with a TNF-α receptor II activator and methods of treating the subjects identified as likely to respond to the treatment. The invention also provides methods for treating subjects that are determined to be unlikely to respond to TNF-α receptor II activator treatment alone.

### BACKGROUND

Autoimmune diseases are believed to involve immune responses to the body's own components that are not observed under normal conditions, which result in a pathological state that causes various tissue disorders and/or functional disorders. Autoimmune diseases are broadly classified into systemic autoimmune diseases and organ-specific autoimmune diseases according to their characteristics. Examples of autoimmune diseases include insulin-dependent diabetes (also known as type 1 diabetes), systemic lupus erythematosus, chronic rheumatoid arthritis, Hashimoto's disease, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, ulcerative colitis, psoriatic arthritis, essential mixed cryoglobulinemia, fibromyalgia-fibromyositis, Graves' disease, Guillain-Barré, hypothyroidism, idiopathic pulmonary fibrosis, idiopathic thrombocytopenia purpura (ITP), IgA nephropathy, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, Wegener's granulomatosis, and autoimmune neurological disorders (e.g., autoimmune Alzheimer's disease, autoimmune Parkinson's disease, or amyotrophic lateral sclerosis).

In particular, type 1 diabetes is a severe, childhood, autoimmune disease, characterized by insulin deficiency that prevents normal regulation of blood glucose levels. Insulin is a peptide hormone produced by β cells within the islets of Langerhans of the pancreas (β-islet cells). Insulin promotes glucose utilization, protein synthesis, formation and storage of neutral lipids, and is the primary source of energy for brain and muscle tissue. Type 1 diabetes is caused by an autoimmune reaction that results in complete destruction of the β-islet cells of the pancreas, which eliminates insulin production and eventually results in hyperglycemia and ketoacidosis. Insulin injection therapy has been useful in preventing severe hyperglycemia and ketoacidosis, but fails to completely normalize blood glucose levels. Although insulin injection therapy has been quite successful, it does not prevent the premature vascular deterioration that is the leading cause of morbidity among diabetics today. Diabetes-related vascular deterioration, which includes both microvascular deterioration and acceleration of atherosclerosis, can eventually cause renal failure, retinal deterioration, angina pectoris, myocardial infarction, peripheral neuropathy, and atherosclerosis.

C-peptide, or connecting peptide, is a short 31 amino-acid protein that connects the A-chain and B-chain of insulin within the proinsulin molecule. β-Islet cells of the pancreas secrete preproinsulin containing an A-chain, a C-peptide, a B-chain, and a signal sequence. The signal sequence is cleaved to yield proinsulin. Subsequently, C-peptide is cleaved to produce the mature insulin protein, which contains the disulfide-linked A-chain and B-chain. Newly diagnosed diabetes patients are often stratified into those having type 1 diabetes and type 2 diabetes based on their C-peptide levels. Measurement of C-peptide levels in a subject constitutes a useful proxy test for insulin production in the body of the subject, as variability of insulin levels within the circulatory system of a subject due to liver metabolism renders the measurement of insulin levels inconclusive. C-peptide levels, on the other hand, are minimally affected by liver metabolism. Consequently, peripheral C-peptide concentrations reflect the secretion of insulin by β-islet cells more accurately than insulin concentration.

HbA1c (glycated hemoglobin or glycosylated hemoglobin) is a form of hemoglobin that is produced in vivo by a non-enzymatic glycation of hemoglobin in plasma. The ratio of HbA1c to non-glycated forms of hemoglobin is directly correlated to the glucose concentration in plasma. Therefore, high levels of HbA1c observed over a prolonged period of time in a sample from a diabetic may be indicative of a serious condition, such as hyperglycemia (e.g., acute hyperglycemia).

Tumor necrosis factor-alpha (TNF-α) is a naturally occurring cytokine that was described in 1975 as the serum factor induced after Bacillus Calmette-Guérin (BCG) injection as a means to fight tumors (Carswell et al., Proc. Natl. Acad. Sci. U.S.A. 72:3666-3670, 1975). The cloning of TNF-α and its two receptors uncovered sequence homology to the genomes of microbial pathogens (e.g., Loetscher et al., Cell 62:351, 1990). This surprising sequence overlap represents a system of intricate microbial responses to modulate host TNF-α secretion and the activity of its receptors (Rahman et al., PloS Pathogens 2:66, 2006).

TNF-α expression is induced by diverse bacteria, parasites, and viruses as a host first line defense to infections. Viruses, such as the Epstein-Barr virus, encode receptors and proteins that even augment TNF-α and TNF-α signaling (Liebowitz, New Engl. J. Med. 338:1461-1463, 1998; Guasparri et al., Blood 111:3813-3821, 2008; Wang et al., Cell 43:831-840, 1985). Alternatively, a variety of viruses have been shown to express proteins that repress TNF-α signaling activity and function in the host (Rahman et al., PloS Pathogens 2:66, 2006). Some evidence suggests that viral infections (e.g., Epstein-Barr virus infections) may cause autoimmune disease (Sairenji et al., Diabetologia 34:33-39, 1991).

Although a number of investigated therapies against autoimmune diseases have demonstrated some palliative or even curative properties, these therapies may not produce consistently beneficial outcomes in all subjects having an autoimmune disease, such as type 1 diabetes. Therefore, there remains a need for methods to accurately identify subjects that are likely to respond to a treatment or a set of treatments prior to treating these subjects, as well as therapies for treating subjects identified as likely to respond to the treatment.

### SUMMARY OF THE INVENTION

In the first aspect, the invention provides a method of determining the likelihood a subject having an autoimmune disease will respond to treatment with a tumor necrosis factor-α (TNF-α) receptor II (TNFR2) activator. The method involves:
(i) contacting an in vitro biological sample comprising a population of CD8⁺ T cells from the subject with a composition comprising a TNFR2 activator; and
(ii) measuring CD8 protein density on the surface of autoreactive CD8⁺ T cells in the population; where reduced CD8 protein density on the surface of the autoreactive CD8⁺ T cells relative to a reference CD8⁺ T cell indicates the subject is likely to respond to the treatment. In some embodiments, the reference CD8⁺ T cell is from a reference sample from a subject having an autoimmune disease and not being treated or pretreated with a TNFR2 activator. In other embodiments, the reference CD8⁺ T cell is from a reference sample from a healthy subject. In certain embodiments, the measuring is performed using anti-CD8 antibody, which may be conjugated to a fluorochrome. In particular embodiments, the biological sample is incubated with dasatinib prior to the contacting (e.g., the biological sample is incubated with dasatinib for at least 4 hours, and/or the biological sample is incubated with dasatinib for at most 48 hours).

In some embodiments of the methods of the invention, the autoimmune disease is type 1 diabetes, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease,

Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, Wegener's granulomatosis, or an autoimmune neurological disorder. In certain embodiments, the autoimmune disease is type 1 diabetes, celiac sprue-dermatitis, Crohn's disease, Graves' disease, hypothyroidism, lupus, multiple sclerosis, psoriasis, rheumatoid arthritis, sarcoidosis, Sjögren's syndrome, or ulcerative colitis. In particular embodiments, the autoimmune neurological disorder is autoimmune-mediated damage to neurons, autoimmune Alzheimer's disease, autoimmune Parkinson's disease, or autoimmune-mediated amyotrophic lateral sclerosis.

In the second aspect, the invention provides a method of determining the likelihood a subject having type 1diabetes will respond to treatment with a tumor necrosis factor-α (TNF-α) receptor II (TNFR2) activator. The method involves contacting an in vitro biological sample from the subject with a device capable of detecting C-peptide in the sample, where a detectable C-peptide level in the sample indicates the subject is likely to respond to the treatment, and where a substantially undetectable C-peptide level indicates the subject is unlikely to respond to the treatment. In some embodiments, a substantially undetectable level of the C-peptide indicates the subject should be excluded from the treatment. In certain embodiments, the substantially undetectable level is a C-peptide level less than about 1.5 pmol/L (e.g., less than about 1.0 pmol/L). In other embodiments, the detectable level is a C-peptide level of greater than about 1.5 pmol/L. In specific embodiments, the subject is identified as likely to respond to the treatment if the concentration of C-peptide is in the range of about 1.5 pmol/L to about 4.0 pmol/L.

In certain embodiments of this aspect of the invention, the contacting is performed prior to the treatment of the subject. The contacting involves:
(i) contacting the sample with the device having an immobilized capture agent on the surface thereof so that the C-peptide in the sample binds to the immobilized capture agent;
(ii) contacting the surface with a detecting binding agent so that the C-peptide binds to the detecting binding agent; and
(iii) measuring the level of the C-peptide in the sample using the detecting binding agent.

In some embodiments, the detecting binding agent includes peroxidase enzyme. In particular embodiments, the measuring involves contacting the surface with a solution containing hydrogen peroxide and a peroxidase substrate.

In some embodiments of this aspect of the invention, the method of the invention further involves measuring a reference HbA1c level in a blood sample from the subject prior to initiating the treatment. The method involves:
(i) measuring HbA1c level in a blood sample taken from the subject after the treatment,
(ii) comparing the HbA1c level to the reference HbA1c level, and
(iii) identifying the subject as in need of a repeat treatment with the one or more TNFR2 activators if the HbA1c level is equal to or greater than the reference HbA1c level.

In particular embodiments of the invention, the blood sample is taken from the subject at least 6 months after the treatment (e.g., at least 1 year after the treatment, at least 2 years after the treatment, at least 3 years after the treatment, or at least 5 years after the treatment).

In particular embodiments of any of the aspects of the invention, the sample contains blood, a blood component, or urine. In certain embodiments, the blood component is serum or plasma. In other embodiments, the sample contains urine.

In some embodiments of any of the aspects of the invention, the TNFR2 activator is Bacillus Calmette-Guérin (BCG), complete Freund's adjuvant, TNF-α, a TNF-α receptor II agonist, a TNF-α mutein, interleukin-1, interleukin-2, tissue plasminogen factor, lipopolysaccharide (LPS), lymphotoxin, or cachectin. In particular embodiments, TNFR2 activator is BCG. In certain embodiments, the autoimmune disease is type 1 diabetes, and the method is used in combination with one or more methods of the second aspect of the invention.

In the third aspect, the invention provides a pharmaceutical composition containing one or more TNFR2 activators for use in treating an autoimmune disease in a subject diagnosed as likely to respond to the treatment. In particular embodiments, a sample from the subject contains a population of T cells having reduced CD8 protein density on the surface thereof following exposure to a TNFR2 activator, relative to CD8protein density on the surface of a reference T cell in a sample from a reference subject (e.g., a healthy subject or a subject having an autoimmune disease and not being treated or pretreated with one or more TNFR2 activators). In some embodiments, the subject is identified as likely to respond to the treatment according to the method of the first aspect of the invention.

In some embodiments of this aspect of the invention, the autoimmune disease is type 1 diabetes, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, Wegener's granulomatosis, or an autoimmune neurological disorder. In some embodiments, the autoimmune disease is type 1 diabetes, celiac sprue-dermatitis, Crohn's disease, Graves' disease, hypothyroidism, lupus, multiple sclerosis, psoriasis, rheumatoid arthritis, sarcoidosis, Sjögren's syndrome, or ulcerative colitis. In particular embodiments, the autoimmune neurological disorder is autoimmune-mediated damage to neurons, autoimmune Alzheimer's disease, autoimmune Parkinson's disease, or autoimmune-mediated amyotrophic lateral sclerosis.

In the fourth aspect, the invention provides a pharmaceutical composition containing one or more TNFR2 activators for use in treating type 1 diabetes in a subject identified as likely to respond to the treatment prior to the treating by determining a level of C-peptide in an in vitro sample from the subject, where a substantially undetectable C-peptide level indicates the subject is unlikely to respond to the treatment, and where a detectable C-peptide level indicates the subject is likely to respond to the treatment. In particular embodiments, the substantially undetectable C-peptide level indicates the subject should be excluded from the treatment. In some embodiments, the substantially undetectable level is a C-peptide level less than 1.5 pmol/L. In other embodiments, the detectable level is a C-peptide less of greater than 1.5 pmol/L. In certain embodiments, the subject is excluded from the treating if a urine sample from the subject exhibits a C-peptide to creatinine ratio of less than about 4.0 pmol/mmol. In yet other embodiments, the subject is identified as likely to respond to the treatment if a urine sample from the subject exhibits a C-peptide to creatinine ratio of greater than or equal to about 4.0 pmol/mmol. In particular embodiments, the subject is identified as likely to respond to the treatment according to the methods of the second aspect.

In some embodiments of this aspect, the pharmaceutical composition is characterized in that HbA1c levels of the subject decrease by at least 0.1% within about 4 years after administration of the composition. In other embodiments, the pharmaceutical composition is characterized in that HbA1c levels of the subject decrease by at least 0.1% within about 3 years after administration of the composition. In specific embodiments, the composition is for use in treating type 1 diabetes in the subject a second or subsequent time, where HbA1c levels in the subject increase or remain the same relative to HbA1c level in the subject prior to the previous treatment. In particular embodiments, the subject is identified as in need of a repeat treatment with one or more TNFR2 activators according to the method of the second aspect. In some embodiments, the subject is a human. In other embodiments, the subject is a long term diabetic.

In some embodiments of any of the aspects of the invention, the one or more TNFR2 activators are selected from Bacillus Calmette-Guérin (BCG), complete Freund's adjuvant, TNF-α, a TNF-α receptor II agonist, a TNF-α mutein, interleukin-1, interleukin-2, tissue plasminogen factor, lipopolysaccharide (LPS), lymphotoxin, and cachectin. In particular embodiments, the one or more TNFR2 activators is BCG. In particular embodiments, the composition contains greater than 2×10⁶ CFU/dose of BCG (e.g., 2.3×10⁶ CFU/dose of BCG). In specific embodiments, the composition contains less than 4×10⁶ CFU/dose of BCG. In some embodiments, the composition contains lyophilized BCG. In particular embodiments, the composition contains saline solution of BCG.

In particular embodiments of any of the aspects of the invention, the composition is administered to the subject one or more times (e.g., two or more times, such as twice). In some embodiments, at least two of the administrations of the composition are spaced at least two weeks apart. In other embodiments, at least two of the administrations of the composition are spaced at least four weeks apart.

In certain embodiments of any of the aspects of the invention, the composition is formulated for administration by a route selected from intradermally, intramuscularly, parenterally, intravenously, intra-arterially, intracranially, subcutaneously, intraorbitally, intraventricularly, intraspinally, intraperitoneally, and intranasally. In specific embodiments, the composition is formulated for intradermal administration. In particular embodiments, the composition is formulated for administration as a saline solution. In some embodiments, the solution has a volume of less than about 0.2 cc per dose, such as a volume of 0.1 cc per dose. In some embodiments, the pharmaceutical composition is formulated for separate administration of two or more TNFR2 activators. In other embodiments, the pharmaceutical composition is formulated for combined administration of two or more TNFR2 activators.

In some embodiments of the pharmaceutical composition of the invention, the pharmaceutical composition is characterized as being capable of inducing expression of TNF-α in the subject. In particular embodiments, the pharmaceutical composition is characterized in that the composition is capable of inducing activation of the NF-κB pathway in an autoreactive immune cell (e.g., autoreactive CD8⁺ T cell) of the subject. In specific embodiments, the pharmaceutical composition is characterized in that the composition causes death of an autoreactive immune cell (e.g., autoreactive CD8⁺ T cell) in the subject. In certain embodiments, the pharmaceutical composition is characterized in that the composition causes expansion of regulatory T cells (e.g., regulatory CD4⁺ T cells) in the subject.

In certain embodiments of any of the aspects of the invention, the pharmaceutical composition is characterized in that the composition prevents a complication from hyperglycemia in the subject. In some embodiments, the complication from hyperglycemia is selected from kidney damage, neurological damage, cardiovascular damage, damage to the retina, damage to the feet, damage to the legs, damage to the heart, and ketoacidosis. In specific embodiments, the composition contains one or more pharmaceutically acceptable carriers or excipients.

### Definitions

By “about,” as used herein, is meant a value that is ±10% of the recited value.

By “antibody,” as used herein, is meant a whole antibody or immunoglobulin and any antigen-binding fragment or single chain thereof. Antibodies, as used herein, can be mammalian (e.g., human or mouse), humanized, chimeric, recombinant, synthetically produced, or naturally isolated. In most mammals, including humans, whole antibodies have at least two heavy (H) chains and two light (L) chains connected by disulfide bonds. Each heavy chain consists of a heavy chain variable region (abbreviated herein as V_{H}) and a heavy chain constant region. The heavy chain constant region consists of three domains, C_{H}1, C_{H}2, and C_{H}3 and a hinge region between C_{H}1 and C_{H}2. Each light chain consists of a light chain variable region (abbreviated herein as V_{L}) and a light chain constant region. The light chain constant region consists of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Antibodies of the present invention include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

By “diabetic,” as used herein, is meant a subject diagnosed with type 1 diabetes. In particular, a long-term diabetic is a subject having type 1 diabetes for at least about 5 years (e.g., at least about 6 years, at least about 7 years, at least about 8 years, at least about 9 year, at least 10 years, at least 11 years, at least about 12 years, at least about 13 years, or at least about 14 years) since the onset of type 1 diabetes.

By “Hox11⁺ splenocyte,” as used herein, is meant a pluripotent CD45⁻ cell expressing the Hox11 gene and found in spleen.

By “immune cell,” as used herein, is meant any cell that is involved in the generation, regulation, or effect of the acquired or innate immune system. Immune cells include, e.g., T cells (e.g., CD4⁺ cells or CD8⁺ cells), B cells, natural killer (NK) cells, macrophages, monocytes and dendritic cells, and neutrophils.

By “mutein,” as used herein, is meant a polypeptide that differs in its amino acid sequence by at least one or more amino acids. For example, a mutein may have an amino acid sequence with greater than 90% but less than 100% sequence identity relative to the amino acid sequence of a reference polypeptide.

By “pharmaceutically acceptable carrier” or “pharmaceutically acceptable excipient,” as used interchangeably herein, is meant any ingredient other than the compounds described herein (for example, a vehicle capable of suspending or dissolving the active compound) and having the properties of being nontoxic and non-inflammatory in a patient. Carriers and excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary carriers and excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, xylitol, water, phosphate-buffered saline (PBS), acetate-buffered saline (ABS), Ringer's solution, dextrose, glycerol, ethanol, or the like and combinations thereof.

By “sample,” as used herein, is meant any specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject. Preferably, the sample is blood, a blood component (e.g., serum or plasma), or urine.

By “subject” or “patient,” as used interchangeably herein, is meant any animal, such as a mammal (e.g., a human). A subject to be treated with a pharmaceutical composition described herein may be one who has been diagnosed by a medical practitioner as having such a condition (e.g., an autoimmune disease, such as type 1 diabetes) or one at risk for developing the condition. Diagnosis may be performed by any technique or method known in the art. One skilled in the art will understand that a subject may have been diagnosed as having an autoimmune disease, such as type 1 diabetes, using a standard test or examination or may have been identified, without examination, as one at high risk due to the presence of one or more risk factors. For type 1 diabetes, such risk factors include, e.g., presence of autoreactive T cells, such as autoreactive CD8⁺ T cells, fasting plasma glucose levels of at least 6.1 mmol/L, plasma glucose levels of at least 11.1 mmol/L two hours after oral glucose load of 75 g, or decreased serum levels of C-peptide. Before treatment with a pharmaceutical composition of the invention, a subject having an autoimmune disease, such as type 1 diabetes, may be subjected to a diagnostic test described herein in order to determine whether the subject is likely to respond to treatment with the pharmaceutical composition of the invention or as unlikely to respond to the treatment.

By “substantially undetectable C-peptide level,” as used herein, is meant that a sample from a subject may have C-peptide levels of less than about 1.5 pmol/L, such as less than about 1.0 pmol/L, or even less than about 0.5 pmol/L. The C-peptide level corresponding to a substantially undetectable C-peptide level does not depend on the feeding state of the subject at the time the sample is obtained. For example, the sample may be from a fasting subject or from a subject that received a stimulus, such as mixed meal tolerance test or stimulation by glucagon test.

By “tumor necrosis factor-α (TNF-α) inducing substance” or “TNF-α inducing substance,” as used interchangeably herein, is meant a composition or a molecule that induces expression of tumor necrosis factor-α (TNF-α) in vivo or in vitro. A TNF-α inducing substance may be Bacillus Calmette-Guérin (BCG), complete Freund's adjuvant, interleukin-1, interleukin-2, tissue plasminogen factor, lipopolysaccharide (LPS), lymphotoxin, or cachectin. Preferably, a TNF-α inducing substance is BCG.

By “TNF-α receptor II (TNFR2) agonist” or “TNFR2 agonist,” as used interchangeably herein, is meant a composition or a molecule that activates TNFR2 upon binding in vivo or in vitro. Examples of TNFR2 agonists include TNFR2 agonist antibodies and TNF muteins that specifically bind and activate TNFR2.

By “TNF-α receptor II (TNFR2) activator” or “TNFR2 activator,” as used interchangeably herein, is meant a composition that directly or indirectly activates TNFR2 in vivo or in vitro. A TNFR2 activator may activate TNFR2 directly, such as by binding TNFR2, or indirectly, such as by inducing the expression of TNF-α. TNFR2 activators include TNF-α, TNF-α inducing substances, and TNFR2 agonists.

### BRIEF DESCRIPTION OF THE DRAWINGS

FIG. 1A is a set of three graphs presenting time course data showing the appearance of EBV-specific T-cells in Subject 1, Subject 2, and Subject 3. In each of the graphs, time point 0 indicates the estimated occurrence of EBV infection based on the timing of the appearance of antibodies directed against EBV.

FIG. 1B is a set of three dot plots of flow cytometry results of analyses of tetramer-stained samples containing EBV-specific autoreactive CD8⁺ T cells and the negative control. The level of non-specific tetramer binding by CD8⁺ T-cells was determined using a tetramer loaded with an irrelevant peptide sequence (Negative Control, left panel). The middle and right panels show examples of positive staining using EBV-specific tetramers.

FIG. 2 is a set of several graphs showing the time course of C-peptide levels in each of three EBV-infected subjects versus a reference population of subjects having type 1 diabetes without active EBV infection. Subjects 1 and 2 showed a transient increase in C-peptide that was statistically significant as compared to the reference population (p<0.04 and p=0.00134, respectively). Subject 3 did not show increased C-peptide levels.

FIG. 3A is a set of graphs quantifying the presence of insulin-B autoreactive T cells and demonstrating an increased presence of circulating insulin-B autoreactive T-cells after EBV infection in type 1 diabetes as compared to the levels of autoreactive T-cells in an uninfected type 1 diabetes subjects. Background fluorescence for peripheral T-cells from the same subjects stained with an irrelevant peptide bearing class I reagent was 0.2%.

FIG. 3B is a set of flow cytometry histograms for samples from subjects having type 1 diabetes with or without EBV infection and quantifying CD8 protein density on the surface of T cells. Reductions in CD8 protein density on the surface of T cells were specific for the newly appearing insulin-B autoreactive T-cells of the EBV-infected subjects. Long-term type 1 diabetics without EBV infection had smaller numbers of peripheral insulin-B autoreactive T-cells, but the density of the CD8 protein on the surface of T cells was in the normal range. Also the EBV-specific T-cells from the EBV-infected subjects also had a normal density of CD8 protein.

### DETAILED DESCRIPTION

I have discovered methods for determining whether subjects having an autoimmune disease will be likely to respond to treatment with a TNF-α receptor II (TNFR2) activator or unlikely to respond to treatment with a TNF-α receptor II (TNFR2) activator. According to the methods of the invention, the presence, in a sample from a subject having an autoimmune disease, of a population of autoreactive CD8⁺ T cells exposed in vivo or in vitro to one or more TNF-α receptor II (TNFR2) activators that exhibit a reduced CD8 protein density on their surface may indicate that the subject is likely to respond to treatment with one or more TNFR2 activators. The reduction in CD8 protein density on the surface of autoreactive CD8⁺ T cells exposed to one or more TNFR2 activators may be relative to the CD8 protein density on the surface of a reference CD8⁺ T cell. The reference CD8⁺ T cell may be from a sample from a subject having an autoimmune disease and not being treated or pretreated in vivo or in vitro with a TNFR2 activator. Alternatively, the reference CD8⁺ T cell may be from a sample from a healthy subject. According to the methods of the invention, the CD8 protein density on the surface of a CD8⁺ T cell may be determined using an anti-CD8 antibody.

I have also discovered methods of discriminating between subjects having type 1 diabetes that are likely to respond to treatment with a TNF-α receptor II (TNFR2) activator and subjects having type 1 diabetes that are not likely to respond to treatment with a TNFR2 activator. I have discovered that C-peptide levels in a subject can be used to assess the likelihood the subject will respond to treatment, in particular, to treatment with a TNFR2 activator. A subject having type 1 diabetes with a detectable level of C-peptide (e.g., a level of greater than or equal to about 1.5 pmol/L) is likely to respond to treatment with a pharmaceutical composition containing a TNFR2 activator. In particular, a response to treatment with a TNFR2 activator may be assessed by detecting an increase in C-peptide levels in the subject following treatment with a TNFR2 activator. On the other hand, a subject having type 1 diabetes with a substantially undetectable level of C-peptide (e.g., a level of less than about 1.5 pmol/L) is unlikely to respond to treatment with a pharmaceutical composition containing a TNFR2 activator (e.g., the subject may be excluded from such therapy or may be administered TNFR2 therapy in combination with a cellular therapy, such as therapy with a pluripotent cell (e.g., a Hox11⁺ cell)).

C-peptide levels may be assayed using a sample from the subject, such as blood, a blood component (serum or plasma), or urine. Measurement of C-peptide levels is possible using any method known in the art, such as enzyme-linked immunosorbent assay (ELISA), which is described in Thermo Scientific Pierce Assay Development Technical Handbook, Version 2, 2011, the disclosure of which is incorporated herein by reference.

The use of a TNFR2 activator in treating, e.g., type 1 diabetics having detectable C-peptide levels is predicated on the discovery that β-islet cells in the pancreas may be restored or regenerated over time if damage to these cells can be reduced (e.g., by reducing the number of autoreactive T cells that target β-islet cells). Subjects having type 1 diabetes with substantially undetectable C-peptide levels may not be capable of restoring or regenerating β-islet cells in the pancreas unlike subjects with detectable C-peptide levels. This is likely due to the loss of many or even substantially all of the β-islet cells as a result of the disease (e.g., a subject with substantially undetectable C-peptide levels likely has an insufficient number of the β-islet cells remaining in their body, and thus would be unable to establish normoglycemia even in the absence of cell injury due to autoreactive immune cells). Moreover, the restoration or regeneration of β-islet cells may be related to the differentiation potential of pluripotent cells (e.g., Hox11⁺ splenocytes) residing in the subject, which may provide a source of cells that can produce a variety of cell types, including β-islet cells. A substantially undetectable C-peptide level in a subject may also indicate that the subject lacks a sufficient number of these pluripotent cells, such as Hox11⁺ splenocytes, to restore or regenerate β-islet cells and to establish normoglycemia.

The beneficial activity of TNFR2 activators in subjects having type 1 diabetes with detectable C-peptide levels relates to their ability to kill autoreactive CD8⁺ T cells in vivo (see, e.g., Ban et al., Proc. Nat. Acad. Sci, USA, 105:13644-13649, 2008, which is incorporated herein by reference), which reduces or minimizes tissue damage caused by these cells (e.g., the loss of β-islet cells). TNFR2 activators also promote expansion of beneficial regulatory CD4⁺ T cells in vivo, which modulates the inflammatory component of the disease. Without being bound by any particular theory, it is believed that TNFR2 agonism activates intracellular NF-κB signaling, which is believed to induce apoptosis in autoreactive T cells, thereby treating type 1 diabetes in subjects (e.g., humans) administered the TNFR2 activator.

### Diagnostic Methods of the Invention

### CD8 Surface Protein Assay

The present invention features methods of identifying a subject (e.g., a human) having an autoimmune disease as being likely to respond to treatment with one or more TNFR2 activators. In some embodiments, the method involves: (i) contacting an in vitro biological sample (e.g., blood) containing one or more CD8⁺ T cells from the subject with a composition containing a TNFR2 activator, and (ii) detecting autoreactive CD8⁺ T cells using an anti-CD8 antibody. A reduced CD8 protein density on the surface of one or more of the autoreactive CD8⁺ T cells relative to a reference CD8⁺ T cell indicates the subject is likely to respond to the treatment. The reference CD8⁺ T cell, such as an autoreactive CD8⁺ T cell, may be from a sample from a subject having an autoimmune disease and not being treated or pretreated in vivo or in vitro with a TNFR2 activator. Alternatively, the reference CD8⁺ T cell (e.g., non-autoreactive CD8⁺ T cell) may be from a sample from a healthy subject.

The sample (e.g., blood) containing one or more autoreactive CD8⁺ T cells for analysis of CD8 protein density on their surface may be obtained from a subject having an autoimmune disease. The sample can be preserved with a tyrosine kinase inhibitor, such as dasatinib (Axon Medchem BV, Groningen, the Netherlands) (see Lissina et al., J. Immunol. Methods, 340:11-24, 2009, which is incorporated herein by reference), which metabolically “freezes” the cells in the sample. The end concentration of dasatinib in the sample may be at least about 10 nM (e.g., at least 50 nM, at least about 100 nM, at least about 200 nM, at least about 500 nM, or at least about 1 uM). To preserve CD8⁺ T cells, the sample containing these cells and the tyrosine kinase inhibitor may be incubated for at least about 4 h (e.g., at least about 6 h, at least about 8 h, at least about 10 h, at least about 12 h, or at least about 14 h) and up to about 48 h (e.g., up to about 40 h, up to about 36 h, up to about 32 h, up to about 28 h, up to about 24 h, or up to about 20 h) prior to conducting a determination of cell-surface CD8 protein density using the cells in the sample. Dasatinib is a metabolic inhibitor that does not alter the cell surface structures. Therefore, cells preserved with dasatinib can be accurately stained even if stored for 4-48 hours.

The sample (whether fresh or preserved with a tyrosine kinase inhibitor, such as dasatinib) may be analyzed, e.g., by contacting the sample with an anti-CD8 antibody conjugated to any fluorochrome known in the art. Non-limiting examples of fluorochromes include FITC, RD1, allophycocyanin (APC), a CF™ dye (Biotium, Hayward, CA), BODIPY (INVITROGEN™ of Life Technologies, Carlsbad, CA), ALEXA FLUOR® (INVITROGEN™ of Life Technologies, Carlsbad, CA), DyLight Fluor (Thermo Scientific Pierce Protein Biology Products, Rockford, IL), ATTO (ATTO-TEC GmbH, Siegen, Germany), FluoProbe (Interchim SA, Motluçon, France), and Abberior Probes (Abberior GmbH, Göttingen, Germany). Methods, such as flow cytometry, may be used to detect fluorescence from a fluorochrome conjugated to an anti-CD8 antibody that is attached to the CD8 protein on the surface of an autoreactive T cell. The intensity of the fluorescence provides a quantitative measure of the CD8 protein density on the surface of the cell.

The detection of a decrease in cell-surface CD8 protein density of a population of autoreactive CD8⁺ T cells, following exposure to a TNFR2 activator, indicates the subject is likely to respond to treatment for an autoimmune disease using the TNFR2 activator. A reduction in cell-surface CD8 protein density on autoreactive CD8⁺ T cells, relative to a reference CD8⁺ T cell, indicates the autoreactive CD8⁺ T cells are undergoing apoptosis resulting from treatment with the TNFR2 activator. The reference CD8⁺ T cell may be a non-autoreactive CD8⁺ T cell from a healthy subject. Alternatively, the reference CD8⁺ T cell may be an autoreactive CD8⁺ T cell that has not been exposed to a TNFR2 activator in vivo or in vitro. Thus, a decrease in CD8 cell-surface density detected using the assay described above indicates a subject is likely to respond to in vivo therapy with a TNFR2 activator, which would be expected to cause autoreactive CD8⁺ T cell death during treatment. The absence of viable autoreactive CD8⁺ T cells would promote a decrease in cellular damage caused by these cells and possibly regeneration of damaged tissues, which may improve the health of the subject.

### C-Peptide Assay

The present invention also features methods of identifying a subject (e.g., a human) having type 1 diabetes as being likely to respond to treatment with one or more TNFR2 activators. The method involves contacting an in vitro biological sample (e.g., blood, a blood component (such as serum or plasma), or urine) from the subject (e.g., a human) with a device capable of detecting C-peptide in the sample. The detection of a level of C-peptide in the sample of greater than about 1.5 pmol/L (e.g., greater than about 2.5 pmol/L) indicates that the subject (e.g., a human) is likely to respond to the treatment. The detection of a level of C-peptide in the sample of less than about 1.5 pmol/L indicates that the subject is unlikely to respond to the treatment (e.g., the subject may be excluded from such therapy or may be administered TNFR2 therapy in combination with a cellular therapy (e.g., a pluripotent cell, such as a Hox11⁺ splenocyte)). The detection of a level of C-peptide in the sample in the range of about 1.5 pmol/L to about 900 pmol/L (e.g., about 500 pmol/L, about 200 pmol/L, about 100 pmol/L, about 50 pmol/L, or about 4.0 pmol/L) indicates that the subject is likely to respond to the treatment. According to the methods of the invention, the subject may be a long term diabetic. A C-peptide level of greater than 1.5 pmol/L indicates the subject is likely to respond to treatment with a TNFR2 activator. A C-peptide level may be measured in either a sample from a fasting subject (fasting C-peptide level) or a sample from a subject in a mixed meal tolerance test or stimulation by glucagon test. A subject would be responsive to treatment with a TNFR2 activator if the treatment causes cell death of autoreactive CD8⁺ T cells and/or expansion or regeneration of endogenous β-islet cells, thereby leading to an increase in insulin levels and a decrease of average plasma glucose levels (e.g., to establish normoglycemia), relative to the levels prior to the treatment.

According to the methods of the invention, the contacting step may be performed by: (i) contacting the sample with the device having an immobilized capture agent (e.g., an antibody that binds C-peptide) on its surface so that C-peptide in the sample binds to the immobilized capture agent; (ii) contacting the surface with a detecting binding agent so that C-peptide binds to the detecting binding agent (e.g., an antibody that binds C-peptide specifically); and (iii) detecting the level of C-peptide in the sample using the detecting binding agent. The detecting binding agent may be specific to C-peptide. The detecting binding agent may be conjugated to a peroxidase enzyme, which can be detected by contacting the surface of the device with a solution of hydrogen peroxide. The solution of hydrogen peroxide may further contain a peroxidase substrate. According to the methods of the invention, any peroxidase substrate known in the art may be used. Some common peroxidase substrates are 2,2′-azino-bis (3-ethylbenzothiazoline-6-sulphonic acid) (ABTS), 3,3′,5,5′-tetramethyl-benzidine (TMB), o-phenylenediamine dihydrochloride (OPD), 10-acetyl-3,7-dihydroxyphenoxazine (ADHP), luminol, disodium p-nitrophenyl phosphate (PNPP), o-nitrophenyl-β-galactopyranosidase (ONPG), QuantaBlu fluorogenic peroxidase substrate (Thermo Scientific Pierce Protein Biolody Products, Rockford, IL), QuantaRed enhanced chemifluorescent peroxidase substrate (Thermo Scientific Pierce Protein Biolody Products, Rockford, IL), SUPERSIGNAL™ ELISA femto maximum sensitivity substrate (Thermo Scientific Pierce Protein Biology Products, Rockford, IL), and SUPERSIGNAL™ ELISA pico chemiluminescent substrate (Thermo Scientific Pierce Protein Biology Products, Rockford, IL). The reaction of peroxidase substrates with peroxidase may be detected spectrophotometrically.

Detection of C-peptide in a sample from a subject may be performed using any assay known in the art. For example, ultrasensitive C-peptide Enzyme-Linked Immunosorbent Assay (ELISA), such as the one manufactured by Mercodia AB (Uppsala, Sweden), provides a solid phase two-site enzyme immunoassay for the quantitative determination of C-peptide in a sample. The immunoassay is based on the direct sandwich technique, in which two monoclonal antibodies are directed against separate antigenic determinants on the C-peptide molecule. During incubation, C-peptide in the sample reacts with anti-C-peptide antibodies bound to the microtitration well. After washing, peroxidase (e.g., horseradish peroxidase) conjugated anti-C-peptide antibodies are added. After a second incubation and a simple washing step, the bound conjugate may be detected by a reaction of hydrogen peroxide with 3,3′,5,5′-tetramethyl-benzidine (TMB). The reaction is stopped by adding acid to give an endpoint that is read spectrophotometrically. Alternatively, after a second incubation the bound conjugate may be detected by any other peroxidase substrate known in the art, such as the substrates listed above.

According to the methods of the invention, a substantially undetectable level of C-peptide in a sample from a subject having type 1 diabetes may indicate that the subject has lost the ability to regenerate β-islet cells, in particular, if the C-peptide level is lower than about 1.5 pmol/L. A substantially undetectable C-peptide level may also indicate that the subject lacks Hox11⁺ splenocytes capable of regenerating β-islet cells.

Long term efficacy of type 1 diabetes treatment with a TNFR2 activator may be gauged by the ability of the subject to regenerate β-islet cells and/or to reduce the incidence of hyperglycemia and hypoglycemia relative to the incidence of hyperglycemia and hypoglycemia in the subject before the treatment (e.g., efficacy may be gauged by an increase in the duration of time the subject is in a normoglycemic state). The treatment involves administering to the subject a pharmaceutical composition of the invention, such as a composition containing one or more TNFR2 activators, a composition containing one or more pluripotent cells (e.g., Hox11⁺ splenocytes), or a combination thereof. C-peptide levels may be used as a proxy to assess the efficacy of treatment with a pharmaceutical composition of the invention in a treated subject. A change (e.g., an increase) in C-peptide levels in a subject treated with a pharmaceutical composition of the invention indicates the subject has responded to the treatment. An increase in the C-peptide levels in the subject of about 1% (e.g., about 2%, about 5%, about 10%, about 20%, about 30%, about 40%, about 50%, about 100%, about 200%, about 500%, about 1000%, about 2000%, about 5000%, or about 7000%), at least 1 month (e.g., at least 3 month, at least 6 months, at least 1 year, at least 2 years, at least 3 years, at least 4 years, at least 5 years, or at least 6 years) after treatment of the subject, may indicate the successful treatment of the subject and that no repeat treatment of the subject is necessary. A decrease or a lack of a change in the C-peptide levels in a subject, at least 1 month (e.g., at least 3 month, at least 6 months, at least 1 year, at least 2 years, at least 3 years, at least 4 years, at least 5 years, or at least 6 years) after treatment of the subject, may indicate that the subject is in need of a repeat administration of a pharmaceutical composition of the invention. C-peptide levels can be detected at least one month (e.g., at least 3 month, at least 6 months, at least 1 year, at least 2 years, at least 3 years, at least 4 years, at least 5 years, or at least 6 years) after treatment of the subject. Changes in the C-peptide levels may be assessed relative to a reference C-peptide level. The reference C-peptide level may be a C-peptide level detected in a sample taken from the subject prior to a first (or subsequent) treatment with a pharmaceutical composition of the invention.

### HbA1c Assay

The invention also features a method of determining whether a subject is or will likely be responsive to a treatment with a TNFR2 activator (alone or in combination with a composition that includes one or more pluripotent cells, such as Hox11⁺ splenocytes) by measuring the HbA1c level in a blood sample from a subject prior to, during, or after treatment of the subject. HbA1c levels measured prior to treatment of the subject can be used as a reference HbA1c level. Additional measurements of HbA1c levels in a blood sample taken from the subject may be performed at least 1 month (e.g., at least 3 month, at least 6 months, at least 1 year, at least 2 years, at least 3 years, at least 4 years, at least 5 years, or at least 6 years) after treatment of the subject. The methods of the invention may further include comparing the HbA1c level measured after the treatment to the reference HbA1c level, and identifying the subject as in need of a repeat treatment with a pharmaceutical composition of the invention if the HbA1c level is equal to or greater than the reference HbA1c level. After administration of one or more TNFR2 activators, HbA1c levels may be measured in a sample from the subject to determine an endpoint and to assess the long-term success of the treatment. A decrease of at least about 0.1% (e.g., 0.15%, 0.2%, 0.25%, 0.3%, 0.4%, or 0.5%) in HbA1c levels relative to the reference HbA1c level would be indicative of the likely long-term success of the treatment. An increase of at least about 0.1% (e.g., 0.2%, 0.3%, 0.4%, or 0.5%) in HbA1c levels over the reference HbA1c level may indicate that one or more repeat treatments with a pharmaceutical composition of the invention should be given to the subject.

HbA1c level in a blood sample can be measured according to methods known in the art, such as high-performance liquid chromatography, immunoassay, enzymatic assay (Direct Enzymatic HbA1c Assay, Diazyme Laboratories, Poway, CA), capillary electrophoresis (Sebia, Norcross, GA), or boronate affinity chromatography (Trinity Biotech Plc, Bray, Ireland). Methods of measuring HbA1c in a sample from a subject are described in, e.g., Little et al., Clin. Chem. 54:1277-1282, 2008, which is incorporated herein by reference. Standard metrics used to describe HbA1c levels are described in, e.g., Goodall, I., Clin. Biochem. Rev. 26:5-20, 2005, which is incorporated herein by reference.

### Pharmaceutical Compositions of the Invention

The present invention also features pharmaceutical compositions containing one or more TNFR2 activators for use in treating a subject (e.g., a human) having an autoimmune disease (e.g., type 1 diabetes) and, in particular, in treating a subject identified by one or more of the diagnostic methods of the invention as likely to respond to the treatment (e.g., likely to experience CD8⁺ T cell death and/or likely to experience restoration and/or regeneration of tissues damaged by the autoimmune disease (e.g., β-islet cells in subjects with type 1 diabetes)).

A pharmaceutical composition containing one or more TNFR2 activators may be used in treating an autoimmune disease, such as type 1 diabetes, in a subject that is likely to respond to the treatment. A reduction in the density of CD8 protein on the surface of an autoreactive CD8⁺ T cell in a sample from the subject having an autoimmune disease, relative to the density of CD8 protein on the surface of a reference T cell, indicates that the subject is likely to respond to treatment with a TNFR2 activator. The reference T cell may be obtained from the subject to be treated or from a different subject. The reference T cell may be a T cell from a healthy subject or from a subject having an autoimmune disease that has not yet been treated with one or more TNFR2 activators.

Examples of autoimmune diseases that can be treated using a pharmaceutical composition of the invention in a subject identified as likely to respond to treatment according to one or more of the diagnostic methods of the invention include type 1 diabetes, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Devic's disease, Takayasu arteritis, temporal arteritis/giant cell arteritis, ulcerative colitis, uveitis, vasculitis, vitiligo, Wegener's granulomatosis, and an autoimmune neurological disorder. In particular, subjects having type 1 diabetes, celiac sprue-dermatitis, Crohn's disease, Graves' disease, hypothyroidism, lupus, multiple sclerosis, psoriasis, rheumatoid arthritis, sarcoidosis, Sjögren's syndrome, or ulcerative colitis, that are identified as likely to respond to therapy according to the diagnostic methods of the invention can be treated. An autoimmune neurological disorder may be an autoimmune-mediated disorder that causes damage to neurons, such as motoneurons. Particular autoimmune neurological disorders include autoimmune Alzheimer's disease, autoimmune Parkinson's disease, and autoimmune-mediated amyotrophic lateral sclerosis (ALS).

Substantially undetectable C-peptide levels in a sample (e.g., blood, a blood component (such as serum or plasma), or urine) from a subject having type 1 diabetes indicate that the subject as unlikely to respond to treatment with a pharmaceutical composition containing one or more TNFR2 activator (e.g., the subject may be excluded from such therapy or may be administered TNFR2 therapy in combination with a cellular therapy, such as a pluripotent cell (e.g., a Hox11⁺ cell)). Detectable C-peptide levels (e.g., levels greater than about 1.5 pmol/L) in a sample from a subject having type 1 diabetes indicate that the subject is likely to respond to the treatment with a pharmaceutical composition containing one or more TNFR2 activators. The subject may be a long term diabetic. The compositions of the invention may be used to treat the subject having type 1 diabetes and identified as likely to respond to the treatment with a TNFR2 activator according to any one or more of the methods of the invention.

In addition, a C-peptide to creatinine ratio of less than about 4 pmol/mmol in a urine sample from a subject having type 1 diabetes may also indicate that the subject is unlikely to respond to the treatment with a pharmaceutical composition containing one or more TNFR2 activator (e.g., the subject may be excluded from such therapy or may be administered TNFR2 therapy in combination with a cellular therapy, such as a pluripotent cell (e.g., a Hox11⁺ cell)). A C-peptide to creatinine ratio of greater than about 4 pmol/mmol in a urine sample from a subject having type 1 diabetes may indicate that a subject is likely to respond to the treatment with one or more TNFR2 activators. A C-peptide to creatinine ratio of from about 4 pmol/mmol to about 11 pmol/mmol in a urine sample from a subject having type 1 diabetes may indicate that a subject is likely to respond to the treatment with one or more TNFR2 activators. The C-peptide to creatinine ratios in urine samples can be determined as described in Besser et al., (Diabetes Care 34:607-609, 2011), which is incorporated herein by reference. Those subjects identified as being likely to respond to treatment with a TNFR2 activator (e.g., BCG) may subsequently be treated one or more times with a TNFR2 activator, such as BCG.

Pharmaceutical compositions of the invention may contain one or more TNFR2 activators include Bacillus Calmette-Guérin (BCG), complete Freund's adjuvant, TNF-α, a TNF-α receptor II agonist (non-limiting examples of TNFR2 agonists, such as antibodies, are described in U.S. Pat. No. 7,582,313, U.S. Pat. No. 8,017,392, and U.S. Pat. No. 8,173,129, which are incorporated herein by reference), a TNF-α mutein (non-limiting examples of TNF-α muteins are described in U.S. Pat. No. 5,597,899, which is incorporated herein by reference), interleukin-1, interleukin-2, tissue plasminogen factor, lipopolysaccharide (LPS), lymphotoxin, and cachectin. Some of the pharmaceutical compositions of the invention may be capable of inducing expression of TNF-α in a subject upon administration. Other compositions of the invention may specifically activate the TNF-α receptor II (as an agonist) and/or induce activation of the NF-κB pathway in an autoreactive immune cell (e.g., autoreactive CD8⁺ T cell) of the subject upon administration. Preferably, administration of the composition of the invention also causes death of one or more autoreactive immune cells in the subject. The compositions of the invention may also cause expansion of regulatory T cells (e.g., regulatory CD4⁺ T cell) in the subject. The compositions of the invention may also reduce or treat a complication from hyperglycemia (e.g., kidney damage, neurological damage, cardiovascular damage, damage to the retina, damage to the feet, damage to the legs, damage to the heart, or ketoacidosis) in a subject having type 1 diabetes.

A TNFR2-activator-containing pharmaceutical composition of the invention may be used to treat type 1 diabetes in a subject (e.g., a human) a second or subsequent time if, following a first treatment, HbA1c levels in a sample (e.g., blood, a blood component (such as serum or plasma), or urine) from the subject increase or remain the same relative to a reference HbA1c level from the subject prior to the previous treatment. A decrease in HbA1c levels in a treated subject may indicate that the subject is responding to the TNFR2 activator treatment (e.g., the subject may not need a second or subsequent treatment). HbA1c levels in a subject that decrease by at least about 0.1% (e.g., at least about 0.15%, at least about 0.2%, at least about 0.25%, at least about 0.3%, at least about 0.4%, at least about 0.5%, at least about 0.6%, at least about 0.7%, at least about 0.8%, at least about 0.9%, at least about 1.0%) within about 4 years (e.g., within about 3 years) after administration of a TNFR2 activator to the subject indicate the subject is responding to the treatment.

### Cellular Therapies

The identification of a subject having type 1 diabetes with a substantially undetectable C-peptide level (e.g., a C-peptide level of less than about 1.5 pmol/L) according to the methods of the invention may indicate that the subject would benefit from, or is in need of, treatment with β-islet cells or cells capable of regenerating β-islet cells, such as pluripotent cells (e.g., Hox11⁺ splenocytes). Hox11⁺ splenocytes are an endogenous source of cells that can regenerate β-islet cells in a subject in vivo, as is described in International Pub. No. WO 2005/042727, which is incorporated herein by reference. Therefore, a subject having type 1 diabetes may have a long term benefit (e.g., within 4 years, such as within 3 years) from cellular treatment if the treatment with one or more TNFR2 activators kills autoreactive T cells and allows the subject to regenerate endogenous β-islet cells (e.g., through expansion of endogenous β-islet cells or due to differentiation of pluripotent cells, such as Hox11⁺ splenocytes, into β-islet cells). Other types of pluripotent cells that can differentiate into β-islet cells in a subject may be administered to the subject, including, for example, those described in U.S. Pat. Nos. 7,432,104 and 8,008,075, which are incorporated herein by reference. Substantially undetectable C-peptide levels (e.g., less than about 1.5 pmol/L) in a sample from a subject having type 1 diabetes may indicate that the subject has lost the ability to regenerate β-islet cells from, e.g., the Hox11⁺ splenocyte compartment. Thus, the subject may benefit from administration of an exogenous source of β-islet cells.

Non-limiting examples of pluripotent cells that may be administered to a subject in need thereof according to the present invention include those described in, e.g., WO 2002/059278, WO 2003/026584, WO 2005/042727, WO 2006/074308, WO 2012/152717, U.S. Pat. No. 7,432,104, and U.S. Pat. No. 8,008,075, which are incorporated herein by reference. Compositions containing one or more pluripotent cells (e.g., Hox11⁺ splenocytes) may be administered prior to, subsequent to, or concurrently with the TNFR2 activator-containing compositions, or the two compositions may be combined for administration in a single dosage form.

### Formulations of the Pharmaceutical Compositions of the Invention

The pharmaceutical compositions of the invention containing one or more TNFR2 activators may be formulated for administration by any route, e.g., intradermal, intramuscular, parenteral, intravenous, intra-arterial, intracranial, subcutaneous, intraorbital, intraventricular, intraspinal, intraperitoneal, or intranasal. The composition of the invention may be formulated for intradermal administration.

The pharmaceutical composition of the invention may contain a pharmaceutically acceptable carrier or excipient. Such carriers or excipients may be selected from, for example, water, phosphate-buffered saline (PBS), acetate-buffered saline (ABS), Ringer's solution, dextrose, glycerol, ethanol, or the like and combinations thereof. In addition, if desired, a composition for administration to a subject can contain minor amounts of auxiliary substances, such as wetting or emulsifying agents, or pH buffering agents that enhance the effectiveness of the composition.

Also, TNFR2 activators may be administered at multiple different times and/or frequencies. For example, one or more (e.g., 1, 2, 3, 4, or 5, or more) doses of TNFR2 activators may be administered daily, weekly, monthly, or yearly (e.g., twice daily, biweekly, quarterly, bi-annually, or tri-annually).

A TNFR2-activator-containing composition of the invention (e.g., BCG) may be administered one or more times (e.g., two or more times, such as twice) to a subject having type 1 diabetes and identified, according to the diagnostic methods described herein, as likely to respond to treatment with the TNFR2 activator. The TNFR2-activator-containing composition may be administered two or more times, spaced by, e.g., about 1 week (e.g., about 2 weeks, about 3 weeks, about 4 weeks, about 5 weeks, about 6 weeks, about 7 weeks, or about 8 weeks or more) between administrations. In some embodiments, administration of the TNFR2 activator composition may be spaced at least two weeks apart (e.g., four weeks apart).

The TNFR2 activator may be a TNF-α inducing substance, such as BCG. The pharmaceutical composition may contain greater than 2×10⁶ CFU/dose of BCG (e.g., greater than 2.3×10⁶ CFU/dose of BCG). The composition may contain less than 4×10⁶ CFU/dose of BCG. The composition may contain from about 2×10⁶ to about 2.5×10⁶, from about 2.5×10⁶ to about 3×10⁶ CFU/dose of BCG, from about 3×10⁶ to about 3.5×10⁶ CFU/dose of BCG, or from about 3.5×10⁶ to about 4×10⁶ CFU/dose of BCG. The composition may contain from about 2×10⁶ to about 4×10⁶ CFU/dose of BCG. The composition may contain from about 2.3×10⁶ to about 4×10⁶ CFU/dose of BCG. The composition may contain from about 2.5×10⁶ to about 4×10⁶ CFU/dose of BCG. In an embodiment, the BCG composition may be lyophilized. Alternatively, the compositions may contain a saline solution of BCG. The saline solution of BCG may be made by reconstituting lyophilized BCG in saline solution. The solution may have a volume of less than about 0.2 cc per dose (e.g., about 0.1 cc per dose).

A composition of the invention containing one or more pluripotent cells may be administered after the subject is identified as being unlikely to respond to treatment with a TNFR2 activator. The compositions containing pluripotent cells administered to a subject in need thereof may be formulated for administration by any route, e.g., intradermal, intramuscular, parenteral, intravenous, intra-arterial, intracranial, subcutaneous, intraorbital, intraventricular, intraspinal, intraperitoneal, or intranasal. Preferably, the composition containing one or more pluripotent cells is administered parenterally, such as through injection (e.g., intravenous or intramuscular) or surgical transplantation. The compositions containing pluripotent cells may be administered to a subject in need thereof one or more times (e.g., 1, 2, 3, 4, or 5, or more). The one or more administrations may be spaced by a week, 2 weeks, 3 weeks, 4 weeks, 5 weeks, 6 weeks, 2 months, 6 months, 1 year, 1.5 years, 2 years, or 3 years or more. Administration of a TNFR2 activator may be performed a week, 2 weeks, 3 weeks, 4 weeks, 5 weeks, 6 weeks, 2 months, 6 months, 1 year, 1.5 years, 2 years, or 3 years or more after administration of a composition containing pluripotent cells. Alternatively, the TNFR2 activator may be administered concurrently with, or subsequently to, administration of a composition containing pluripotent cells by any route described above (e.g., the routed may be the same or different), or the compositions may be combined for administration in a single dosage form.

### Kits of the Invention

### Kits for CD8 Surface Protein Assay

The invention provides kits including a container for a sample from a subject, a preservative (e.g., dasatinib, such as a sufficient quantity to produce an at least about 10 nM (e.g., at least about 50 nM, at least about 100 nM, at least about 200 nM, at least about 500 nM, or at least about 1 uM) final solution of dasatinib in a sample; e.g., a 1 mL solution having at least about 10 mM dasatinib or a container with at least about 1.0 μg to about 25 mg of dasatinib in solid form), instruments for collecting the sample and/or placing the sample in the container, and instructions for collecting and preserving the sample. The kit may further contain a CD8 antibody conjugated to a fluorochrome and/or a TNFR2 activator (e.g., Bacillus Calmette-Guérin (BCG), complete Freund's adjuvant, TNF-α, a TNF-α receptor II agonist, a TNF-α mutein, interleukin-1, interleukin-2, tissue plasminogen factor, lipopolysaccharide (LPS), lymphotoxin, or cachectin). The instructions in the kits described above may also explain how a practitioner (e.g., a physician, a nurse, or a laboratory assistant) may use the sample from the subject to determine whether the subject is likely to respond to treatment with a TNFR2 activator or not. The kits may further contain an implement for collection and transfer of the sample into the container (e.g., a needle or a catheter), components for keeping the cells at a defined temperature (e.g., between about 4° C. and about 27° C.), such as a coolant (e.g., an ice pack, dry ice, cooling pouch, or cooling plates). The kit may also include a cold box or insulated carrier for transport of the cell sample, if necessary.

### Kits for C-Peptide Detection

The invention also provides kits including a device having a capture agent (e.g., an antibody that binds C-peptide) immobilized on its surface, a detecting binding agent conjugated to a peroxidase enzyme (e.g., an antibody that binds C-peptide selectively), a peroxidase substrate, and instructions explaining how a practitioner (e.g., a physician, a nurse, or a laboratory assistant) may use the contents of the kit for analysis of a sample from a subject to determine whether the subject is likely to respond to treatment with a TNFR2 activator or not.

### Kits for HbA1c Assay

The invention also provides kits including a container for storing a sample from a subject and instructions for determining HbA1c levels in the sample and using the HbA1c levels to determine if the subject requires administration of a TNFR2-activator-containing composition or not.

The following examples are meant to illustrate the invention. They are not meant to limit the invention in any way.

### EXAMPLES

### Example 1. Identification of Subjects Having Type 1 Diabetes as Likely to Respond to Treatment with a TNFR2 Activator

Prior to treatment with a pharmaceutical composition of the invention (e.g., a TNFR2 activator), a subject (e.g., human) may be tested to determine whether the subject is likely to respond to the treatment. The test may be performed using a sample (e.g., blood, a blood component, or urine) collected from the subject. For example, to prepare a serum sample, blood may be collected by venipuncture, allowed to clot, and serum may be separated by centrifugation. To prepare a plasma sample, blood may be collected by venipuncture into tubes containing heparin or EDTA as anticoagulant; a plasma fraction may then be separated from the samples. To prepare a urine sample, a 24 hour urine sample (without preservative) may be collected. Cellular debris may be removed from the sample prior to testing by either filtration or centrifugation.

The prepared sample from the subject may then be assayed using, e.g., a Mercodia ULTRASENSITIVE™ C-peptide ELISA kit (manufactured by Mercodia AB, Uppsala, Sweden) to give C-peptide levels in the sample from the subject. The subject is identified as being unlikely to respond to the treatment with a TNFR2 activator if the measured C-peptide levels are lower than about 1.5 pmol/L. A subject identified as having a substantially undetectable C-peptide level may be excluded from treatment with a TNFR2-activator-containing composition. In particular, such a subject may be determined as having an insufficient number of endogenous β-islet cells remaining or an insufficient amount of pluripotent cells, such as Hox11⁺ splenocytes, remaining to differentiate into β-islet cells, and therefore unlikely to exhibit a restoration of, or improvement in, normoglycemia following therapy with a TNFR2 activator. The determination that a subject has a substantially undetectable C-peptide level may also indicate that the subject would benefit from transplantation of β-islet cells or pluripotent cells, such as Hox11⁺ splenocytes, that can differentiate into β-islet cells. The subject may be determined as likely to respond to treatment with a TNFR2 activator if the measured C-peptide levels are greater than about 1.5 pmol/L; this subject may be treated with a TNFR2 activator composition of the invention with an expectation that such therapy will result in regeneration of β-islet cells and/or restoration of, or improvement in, normoglycemia.

The test may be repeated one or more times to correct for possible single measurement variability of data.

### Example 2. Treatment of Type 1 Diabetes in a Human with a Pharmaceutical Composition of the Invention

Prior to treatment with a TNFR2 activator (e.g., BCG), a sample (e.g., blood) may be taken from the subject identified in Example 1 as likely to respond to the treatment. The sample may be analyzed for HbA1c levels to determine the baseline HbA1c level for the study.

A pharmaceutical composition containing BCG reconstituted in saline may be administered to the subject as two intradermal injections four weeks apart in a dose containing 1.0-2.3×10⁶ CFU/administration (volume=0.1 cc/administration). After the treatment, the subject may be monitored over time by testing samples obtained from the subject using functional assays (autoreactive CD8⁺ T cell assay and regulatory CD4⁺ T cell assay), which are described further below.

After the treatment, 1 year (e.g., 2 years, 3, years, 4, years, 5 years, or 6 years) after administration of the TNFR2 activator, such as BCG, a sample from the subject may be assessed for HbA1c levels to determine the endpoint and to confirm the long-term success of the treatment. A decrease of at least about 0.1% (e.g., 0.15%, 0.2%, 0.25%, 0.3%, 0.4%, or 0.5%) in HbA1c levels relative to the baseline HbA1c level would be indicative of a long-term success of the treatment.

The subject may be continued to be monitored for changes in HbA1c levels. An increase of at least about 0.1% (e.g., 0.2%, 0.3%, 0.4%, or 0.5%) in HbA1c levels over the baseline HbA1c level may indicate that the subject should be administered the pharmaceutical composition of the invention again.

### Immune Monitoring

### 1. T Cell Assays

CD4⁺ and CD8⁺ T cells can be isolated from fresh human blood within 1.5 h of venipuncture using Dynal CD4 positive isolation kit and Dynal CD8 positive isolation kit (Invitrogen, Carlsbad, CA). This method is unique in yielding cells that are both free of magnetic particles and free of the positive selection with the antibody.

### 2. Detection of Autoreactive CD8⁺ T Cells in Type 1 Diabetes

Highly purified, viable and high yield CD8⁺ T cells can be utilized for tetramer staining, as previously described (Verginis et al., Proc. Natl. Acad. Sci. U.S.A. 105:3479-3484, 2008). Tetramers are diagnostic reagents that are composed of the binding region of specific HLA class 1 proteins with loaded peptides in the exterior binding groves. The tetramers are then made fluorescent and act as diagnostic reagents that can bind to T cells with specific reactivity to the presented peptide fragment. For detection of autoreactive T cells to insulin, tetramers to HLA *0210 insulin beta 10-18 with a fragment of HLVEALYLV (SEQ ID NO: 1; Beckman Coulter #T02001) can be used. For negative control tetramers, the following tetramer reagents can be used: HLA *0201 Her-2/neu with a sequence to KIFGSLAFL (SEQ ID NO: 2; Beckman Coulter #T02001), a breast cancer peptide, HLA *0201 null without a non-specific peptide fragment (Beckman Coulter #T01010) and/or a tetramer to the CMV virus HLA-A *0201 CMGPP65 with a sequence of NLVPMVATV (SEQ ID NO: 3; Beckman Coulter #T01009).

Tetramer reagent staining can be conducted both after 12 h of culture at 26° C. followed by 6 h at 37° C. and/or after 1 h rest at 26° C. followed by 12 h at 37° C. The cells can be then stained with Sytox-green (MBL International Co., Woburn, MA) and/or CD8 antibodies (BD Biosciences, San Jose, CA). All cells can be stained at 4° C. in the dark for 30 minutes and then can be washed twice in Hank's buffer with 2% heat-inactivated bovine serum. On average, 100,000 highly pure CD8⁺ T cells can be analyzed to ensure clear data points and to allow for the detection of the rare autoreactive T cells. All cells can be fresh to prevent fixation artifacts and to allow for the quantification of dead versus viable cells. Cell viability can be quantified by either of two stains that fluorescently label dead cells, Sytox (MBL International Co., Woburn, MA) or propidium iodide (PI).

3. Detection of T_{reg} CD4⁺ Cells in Type 1 Diabetics

Two different methods can be used for the detection of T_{REG} cells. T_{REG} cells can be detected using CD4, CD25^{bright}, and Foxp3 staining or with CD4, CD25^{bright}, and CD127^{low} antibody staining. Briefly, isolated CD4 positive cells can be incubated with CD4-PE-Cy5 (clone RPA-T4) and CD25-PE (clone BC96) antibodies for 20 minutes at room temperature. After washing, cells can be fixed with Foxp3 Fix/Perm solution (Biolegend) for 20 minutes at room temperature. Cells can be washed and permeabilized with Biolegend's Foxp3 Perm Buffer for 15 minutes at room temperature. Cells can be then stained with Foxp3 Alexa Fluor477 antibody (clone 259D) for 30 minutes. Isotype controls can be done for each sample prior to flow cytometric analysis. Alternatively, for detection of T regulatory cells, staining can also be performed with a CD4 antibody (clone RPA-T4, BD Biosciences, San Jose, CA) and an anti-human CD127 antibody (clone hIL-7R-M21, BD Biosciences). Other methods for detecting T_{REG} cells are described in U.S. Ser. No. 61/763,217, which is incorporated herein by reference in its entirety.

### Example 3. Detection of CD8⁺ T Cells in a Sample from a Subject Having EBV Infection

### Materials and Methods

### Subjects

Patients having type 1 diabetes were recruited at the Massachusetts General Hospital. The diabetic patients were routinely screened to characterize the course of disease and to exclude subjects with potentially interfering medical conditions. During screening, three patients were identified as having long-term type 1 diabetes and recent onset of EBV. Diabetics without EBV infection (N=66) were used in this study as a reference population. All patient and control blood was drawn into BD VACUTAINER™ tubes (BD, Franklin Lakes, NJ) containing EDTA.

### Ethics Statement

The study was approved by the Massachusetts General Hospital Institutional Review Board (IRB Protocol No. 2001P-001379). Written consent was obtained from all blood donors.

### Detection of Epitope-Specific CD8⁺ T-Cells

For the detection of specific subpopulations of T-cells with restricted antigen specificity, commercially available HLA Class I reagents loaded with small peptide fragments were utilized. These commercial reagents are commonly referred to as Tetramers (MBL International, DesPlaines IL., formerly Beckman Coulter, Fullerton, CA) or Dextramers (Immudex, Fairfax, VA). The two T-cell detection methods differ in the backbone structures of the detecting reagent but not in the binding specificity to autoreactive T-cells. Tetramer or dextramer reagents are purchased fluorescently labeled for detection of the bound reagent to antigen specific T-cell with a flow cytometer.

For the studies contained in this paper, two types of antigen-specific T-cells were detected, i.e., EBV-specific T-cells (HLA class I loaded peptide; GLCTLVAML (SEQ ID NO: 4)) or insulin-B autoreactive T-cells (HLA class I loaded Insulin-B chain; HLVEALYLV (SEQ ID NO: 1)). For background fluorescence of T-cells a matched HLA class I structure was loaded with an irrelevant peptide (Beckman Coulter, Immudex).

### Isolated CD8⁺ T-Cell Method

For the direct isolation of CD8⁺ T-cells from fresh blood a “Detach-a-bead” CD8 positive isolation kit based on paramagnetic beads coated with anti-CD8 antibody was used (Life Technologies, Carlsbad, CA) (see Burger et al., PLoS One, 6: e22430, 2011, which is incorporated herein by reference). The beads were allowed to attach for 1 hr at room temperature under continuous agitation on a tumbler. The bead/cell complexes were then immobilized using a magnet and any unbound (non-CD8) cells removed by repeated washing with HBSS (Hank's Balanced Salt Solution without calcium and magnesium, Invitrogen, Grand Island, NY) containing 2% FBS (fetal bovine serum). The beads were then detached from the remaining CD8⁺ cells using a Detach-A-Bead reagent supplied in the isolation kit. This reagent is a polyclonal antibody directed against the antigen recognition site of the CD8 antibody coated on the beads. It detaches the antibody/bead complex from the cells by means of competition for the CD8 antibody binding site, essentially leaving a virgin cell.

Isolated CD8⁺ T-cells were then labeled with PE (phycoerythrin) labeled tetramers or dextramers (20 min, RT (room temperature), in the dark) and subsequently with APC-anti-CD8 antibodies (10 minutes at RT in the dark; clone SK1, BD Biosciences, San Jose, CA) to determine purity of the isolated cell preps. The samples were then fixed with HBSS 0.1% formaldehyde buffer, washed with HBSS, and re-suspended in HBSS/0.05% formaldehyde for flow cytometry.

### Whole Blood Method

Blood samples were first washed with 50 volumes of HBSS containing 2% FBS. They were then labeled with the tetramers or dextramers (20 min at RT in the dark) and subsequently with APC-anti-CD8 antibodies (10 minutes at RT in the dark) to enable gating on CD8⁺ T-cells. The samples were then simultaneously lysed and fixed with NH4Cl/formaldehyde buffer, washed with HBSS, and re-suspended in HBSS/formaldehyde for flow cytometry.

### Flow Cytometry

Cells were analyzed using a FACSCalibur flow cytometer (BD Biosciences, San Jose, CA) and data collected in list mode. Data analysis was performed using Cell Quest software (BD Biosciences). Flow gates were set “open” for inclusion of all cells. The open gate included cells of all sizes, but excluded cell debris, red blood cells, fragmented cells, and apoptotic bodies. PE and APC fluorescence were detected in FL2 and FL7, respectively. The percentage CD8⁺ T-cells was defined as the ratio of the number of CD8 positive events and the total number of events in the lymphocyte gate.

### ELISA

C-peptide was determined by ELISA in blood serum of the EBV-infected patients. The ULTRASENSITIVE™ C-peptide ELISA was from Mercodia (Uppsala, Sweden). The kit was used according to the manufacturers' instructions. Serum levels of VCA IgM, Early Antigen D, and EBNA were determined by Massachusetts General Hospital Clinical Laboratory Services.

### Statistics

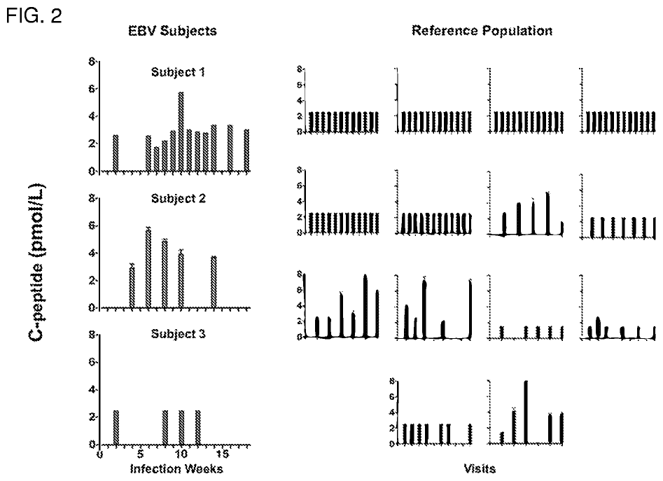

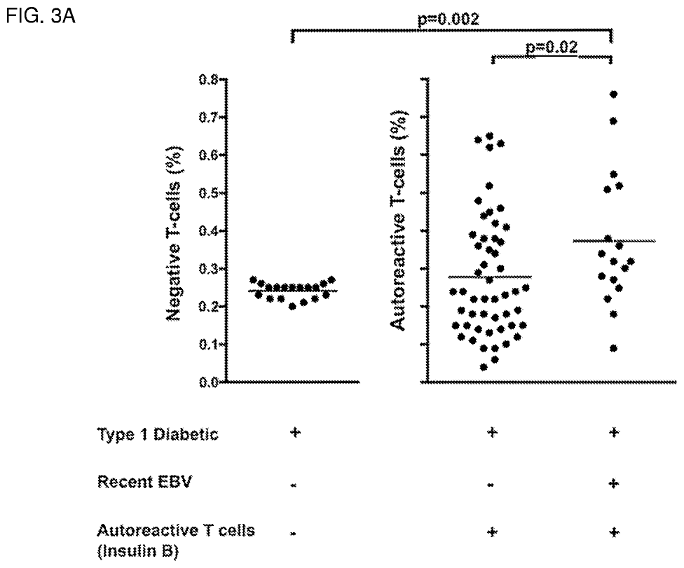

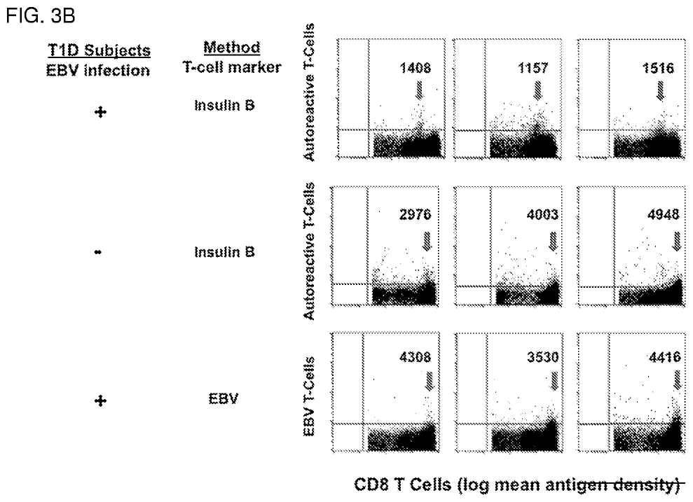

Statistical significance was determined using the unpaired, one-tailed Student's t-test at a confidence level of 0.05 (FIGS. 3A and 2B) or a one sided Kolmogorov-Smirnov test (FIG. 2).

### Results

### Clinical Profiles

In the course of routine visits of type 1 diabetes patients, three patients with recent onset EBV infection were identified, also known clinically as mononucleosis or “mono” (see Table 1). These patients were followed closely for at least 15 weeks after presentation. Recent onset EBV infection offers an opportunity to study the prevalence and traits of autoimmune and EBV-specific T-cells both during and after clinical symptoms of EBV infection. It also offers the opportunity to document the reproducibility of the past observation in a clinical trial of BCG treatment that EBV infection caused a transient increase in insulin production by the pancreas, measured as co-secreted C-peptide (see Faustman et al., PLoS One, 7: e41756, 2012, which is incorporated herein by reference).

TABLE 1
A Summary of Clinical Characteristics for the Three EBV Subjects.
Clinical characteristics of subjects

| | | | Age at | T1D | | |
|---|---|---|---|---|---|---|
| | | Age | Onset | Duration | HbA1c | GADA |
| | Sex | (y) | (y) | (y) | (%) | Status |
| Subject 1 | M | 47 | 17 | 30 | 6.2 | Pos. |
| Subject 2 | F | 17 | 11 | 6 | 8.0 | Pos. |
| Subject 3 | F | 21 | 2 | 19 | 9.6 | Neg. |

The EBV infections were first clinically diagnosed by standard serologic methods and symptoms. The clinical characteristics of these subjects are summarized in Table 1. All subjects had established type 1 diabetes, with durations of 6, 19, and 30 years and elevated HbA1c. Two of the subjects (Subjects 1 and 2) were positive for glutamic acid decarboxylase GAD65 autoantibody, which is an islet-specific marker for type 1 diabetes.

### Time Course of EBV Infection

After presenting with symptoms and a clinical diagnosis of EBV infection, a more detailed EBV antibody serology was performed to determine the details of the time course of the infection and to estimate the time of onset of the infection relative to the sequential blood studies performed here (Table 2).

TABLE 2
Results for EBV antibody testing on blood samples
drawn during the first visit for the three EBV subjects
and correlation with estimated start of EBV infection.
EBV antibody results

| | | | 6-8 Weeks | |
|---|---|---|---|---|
| | 0-6 Weeks, | 4-8 Weeks, | Sustained, | Estimated |
| | VGA IgM | Early Antigen D | EBNA | Duration |
| Subject 1 | Negative | Positive | n/a | 2 weeks |
| Subject 2 | Positive | Positive | Negative | 4 weeks |
| Subject 3 | Positive | Negative | Negative | 2 weeks |

Subclasses of EBV antibodies to different parts of the EBV viral particle peak at different times after infection were detected. A combination of VCA IgM, Early Antigen D, and EBNA was used. These markers peak at 0-6 weeks, 4-8 weeks, and 6-8 weeks after EBV infection, respectively (Table 2). All subsequent data reported in this study, especially timelines of immunologic events, were plotted and extrapolated to an infection date of “0”. For Subject 1, the presentation time for the present study was estimated to be week 2 after infection. For Subject 2, the presentation time was estimated to be week 4 after infection. For Subject 3, the presentation time was estimated to be week 2 after infection.

### Detection of EBV-Specific CD8⁺ T-Cells

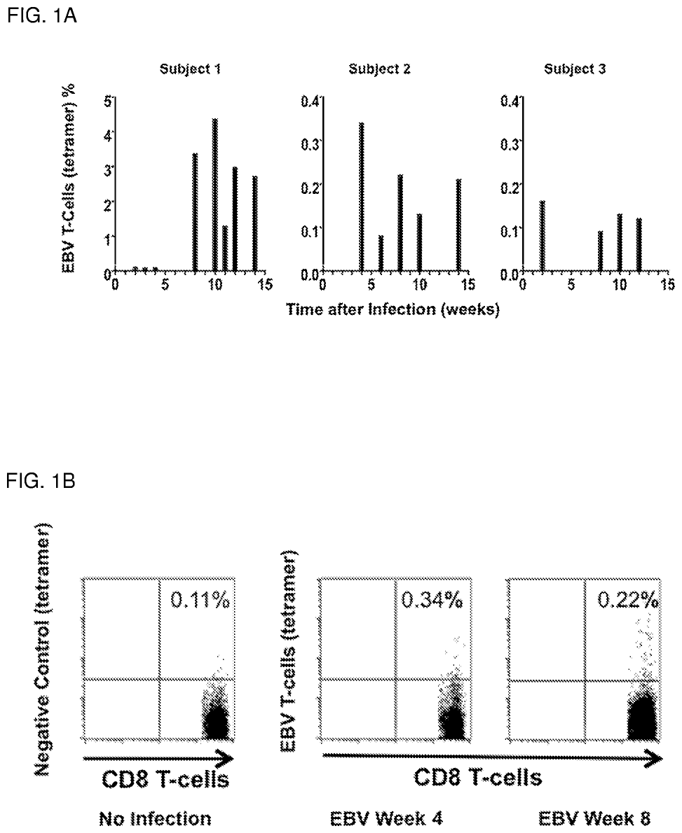

Although the typical clinical method of diagnosing EBV infections involves antibody testing, it is possible in a research setting to confirm infection by direct monitoring of newly created EBV-specific T-cells. EBV-specific tetramers were used, i.e., HLA class I proteins loaded with a synthetic peptide sequence GLCTLVAML (SEQ ID NO: 4). Thus, EBV-specific CD8⁺ T-cells were detected using flow cytometry (FIGS. 1A and 1B). Although a slightly different method for detection of these EBV-specific T-cells was utilized in Subject 1, versus Subjects 2 and 3 (isolated CD8⁺ T-cells versus direct observations of CD8⁺ T-cells in lysed blood), all subjects had an early rise in EBV-specific T-cells above background levels of fluorescence (FIG. 1B).

### Time Course of C-Peptide in EBV-Infected Subjects

Research over the last fifteen years has shown that a systemic increase of TNF, or induction of TNF with either BCG or EBV, results in pancreas regeneration with the recovery of C-peptide (see Kodama et al., Science, 302:1223-1227, 2003; Faustman et al., PLoS One, 7: e41756, 2012; and Faustman, J. Clin. Immunol. 13:1-7, 1993, which are incorporated herein by reference). C-peptide is the protein co-secreted with insulin and a sensitive method to measure insulin secretion from the pancreas in the presence of exogenously administered insulin.

To determine the impact of EBV infections on insulin secretory capacity, serial serum C-peptide levels were monitored for at least 15 weeks after the subjects with EBV presented to the research clinic. A simultaneously studied reference population of non-EBV infected long-term diabetics was similarly monitored for 15 weeks. The monitoring of the reference subjects for fasting morning C-peptide demonstrated the subject and assay variability.

C-peptide in EBV-infected Subjects 1 and 2 showed a statistically significant increase of p=0.04 and p=0.0013 over that in the reference population, respectively. Subject 3 showed no significant increase in pancreatic C-peptide (FIG. 2).

### Impact of EBV on Insulin-B Autoreactive T Cells in Type 1 Diabetics

Previous data show that treatment of NOD mice or diabetic humans with BCG (or the non-cGMP equivalent Complete Freund's Adjuvant (CFA)) results in a transient increase in dead autoreactive T-cells. In the NOD mouse, pancreas-residing autoreactive T-cells are observed to undergo apoptosis directly on top of the insulin-secreting islets in the pancreas with TNF, BCG, or CFA (see Kuhtreiber et al., J. Mol. Endocrinol. 31:373-399, 2003, which is incorporated herein by reference). In humans, the impact of TNF on autoreactive T-cells can be monitored by the rapid release of dead autoreactive T-cells into the circulation (see Faustman et al., PLoS One, 7: e41756, 2012, which is incorporated herein by reference). Increased TNF or TNFR2 agonistic antibodies are known to cause apoptosis of murine and human diabetic autoreactive T-cells in culture (see Ban et al., Proc. Nat. Acad. Sci. USA, 105:13644-13649, 2008, which is incorporated herein by reference).

To determine whether EBV infection similarly kills or injures autoreactive T-cells, insulin-B autoreactive CD8⁺ T-cells in the peripheral blood of the three recently EBV-infected subjects were detected and compared to uninfected long-term diabetic subjects. Tracking of insulin-B autoreactive T-cells in long-term diabetics is feasible. With sensitive monitoring methods about 41% (21 out of 51) of randomly recruited long-term type 1 subjects had detectable insulin-B autoreactive T-cells (0.28% to 0.65%, FIG. 3A). The same type 1 diabetic CD8⁺ T-cells stained with negative dextramer reagents exhibited a background signal range of 0.19% to 0.27% (FIG. 3A).

As previously shown for long-term diabetics exposed to TNF inducing infections, the EBV infected subjects exhibited an overabundance of insulin-B autoreactive T-cells in peripheral blood after infection. The mean percentage of insulin-B autoreactive T-cells in EBV-infected subjects was greater than that in the negative background staining (p=0.002), as well as in a matched population of uninfected reference diabetics (p=0.02). This finding supports the conclusion that infections that boost TNF result in the release of these autoreactive T-cells into the circulation.

### Newly Released Insulin-B Autoreactive T-Cells Have Abnormally Low CD8⁺ Density

A further analysis of the insulin-B autoreactive T-cells in long-term diabetics infected with EBV virus revealed some additional distinctive features of the antigen-specific CD8⁺ T-cells (FIG. 3B). Not only were there more insulin-B autoreactive CD8⁺ T-cells in the circulation after EBV infection, but also the density of the CD8 marker on the cells was dramatically lower. As shown in FIG. 3B, in three characteristic histograms, the CD8 mean log fluorescent density was 1408, 1157, and 1516 for the insulin-B autoreactive T-cells in EBV-infected subjects. In contrast, for insulin-B autoreactive T-cells from uninfected long-term diabetic subjects, the mean log antigen density of the CD8 marker was 2976, 4003, and 4948. The lower density of CD8 proteins in the EBV-infected subjects was not a generalized trend for all antigen-specific T-cells but specific for only the autoreactive T-cells. For these same infected subjects, the log CD8⁺ T-cell density was normal for the EBV-specific T-cells. The CD8 protein density on the cell surface was 4308, 3530, and 4416, values that are similar to the density of the CD8 proteins on insulin-B autoreactive T-cells of uninfected diabetics (FIG. 3B). Loss of CD8⁺ marker is indicative of cellular damage or apoptosis of T-cells (see Diaz et al., J. Leukoc. Biol., 76:609-615, 2004, which is incorporated herein by reference). Thus, the loss of the CD8 protein on the surface of autoreactive T cells in response to a TNFR2 activator, such as TNF-α or BCG, can be used as a proxy to diagnose the likelihood a subject with an autoimmune disease can be treated using a TNFR2 activator.

### Other Embodiments

All publications, patents, and patent applications mentioned in the above specification are hereby incorporated by reference. Various modifications and variations of the described device and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are within the following claims.

1. A method of treating type 1 diabetes in a human subject in need thereof, wherein the subject has previously been administered at least one dose of Bacillus Calmette-Guerin (BCG), the method comprising administering at least a second dose of BCG to the subject more than 1 year later.

2. The method of claim 1, wherein, prior to administration of the BCG, the method further comprises measuring a reference glycated hemoglobin (HbA1c) level in a blood sample from said subject.

3. The method of claim 2, wherein the subject exhibits an increase in HbA1c levels in a blood sample from the subject of at least 0.1% relative to a reference level of HbA1c.

4. The method of claim 1, wherein, prior to administration of the BCG, the method further comprises measuring a reference C-peptide level in a blood sample from said subject.

5. The method of claim 4, wherein the subject exhibits a reduction in C-peptide levels in a blood sample from the subject relative to a reference level of C-peptide.

6. The method of claim 4, wherein C-peptide levels are measured by contacting a blood sample from the subject with a device capable of detecting C-peptide in the blood sample.

7. The method of claim 6, wherein said device comprises an immobilized capture agent on the surface thereof that binds said C-peptide in said sample.

8. The method of claim 4, wherein the C-peptide level is determined when the subject is in a fasting state.

9. The method of claim 4, wherein the C-peptide level is determined when the subject is stimulated by administration of glucagon.

10. The method of claim 1, wherein BCG is administered intradermally to the subject.

11. The method of claim 1, wherein said dose comprises an amount in a range of about 2×10⁶ CFU to about 4×10⁶ CFU of BCG.

12. The method of claim 1, wherein the second dose of BCG is administered to the subject at least 2 years apart.

13. The method of claim 1, wherein the second dose of BCG is administered to the subject at least 3 years apart.

14. The method of claim 1, wherein the subject has a C-peptide level in blood of less than 4.0 pmol/L.
